SEQUENCE LISTING (19 sequences)

SEQ ID NO: 1 - DNA, 393 bp, Artificial
  Analog #1
aacgatcgccacatgattagaatgcgtgagcttatagatgtggttgatcagctgaaaaattatgtgaatgacttggtccctcagtttctgccagctccagaagatatcgagacaaactgtgagtggtcagcttttacctgttttcagaaggcccaactaaagtcagcacagacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaatacctagaaagatacaaatcacttctccaaaagatggtgcatcagcatctgtcctccagaacacacggaagtgaagagacc

SEQ ID NO: 2 - protein, 131 aa, Artificial
  Analog #1
NDRHMIRMRELIDVVDQLKNYVNDLVPQFLPAPEDIETNCEWSAFTCFQKAQLKSAQTGNNERIIQVSIKKLKRKPPTTNAGRRNKHRLTCPSCDTYEKKPPKEYLERYKSLLQKMVHQHLSSRTHGSEET

SEQ ID NO: 3 - DNA, 393 bp, Artificial
  Analog #2
aacgatcgccacatgattagaatgcgtcaacttatagatgtggttgatcagctgaaaaattatgtgaatgacttggtccctcagtttctgccagctccagaagatatcgagacaaactgtgagtggtcagcttttacctgttttcagaaggcccaactaaagtcagcacagacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaatacctagaaagatacaaatcacttctccaaaagatggtgcatcagcatctgtcctccagaacacacggaagtgaagagacc

SEQ ID NO: 4 - protein, 131 aa, Artificial
  Analog #2
NDRHMIRMRQLIDVVDQLKNYVNDLVPQFLPAPEDIETNCEWSAFTCFQKAQLKSAQTGNNERIIQVSIKKLKRKPPTTNAGRRNKHRLTCPSCDTYEKKPPKEYLERYKSLLQKMVHQHLSSRTHGSEET

SEQ ID NO: 5 - DNA, 393 bp, Artificial
  Analog #3
aacgatcgccacatgattagaatgcgtgagcttatagatgtggttgatcagctgaaaaattatgtgaatgacttggtccctcagtttctgccagctccagaagatatcgagacaaactgtgagtggtcagcttttacctgttttcagaaggcccaactaaagtcagcacagacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaatacctagaaagattcaaatcacttctccaaaagatggtgcatcagcatctgtcctccagaacacacggaagtgaagagacc

SEQ ID NO: 6 - protein, 131 aa, Artificial
  Analog #3
NDRHMIRMRELIDVVDQLKNYVNDLVPQFLPAPEDIETNCEWSAFTCFQKAQLKSAQTGNNERIIQVSIKKLKRKPPTTNAGRRNKHRLTCPSCDTYEKKPPKEYLERFKSLLQKMVHQHLSSRTHGSEET

SEQ ID NO: 7 - DNA, 393 bp, Artificial
  Analog #4
aacgatcgccacatgattagaatgcgtgagcttatagatgtggttgatcagctgaaaaattatgtgaatgacttggtccctcagtttctgccagctccagaagatatcgagacaaactgtgagtggtcagcttttacctgttttcagaaggcccaactaaagtcagcacagacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaattcctagaaagatacaaatcacttctccaaaagatggtgcatcagcatctgtcctccagaacacacggaagtgaagagacc

SEQ ID NO: 8 - protein, 131 aa, Artificial
  Analog #4
NDRHMIRMRELIDVVDQLKNYINDLVPQFLPAPEDIETNCEWSAFTCFQKAQLKSAQTGNNERIIQVSIKKLKRKPPTTNAGRRNKHRLTCPSCDTYEKKPPKEFLERYKSLLQKMVHQHLSSRTHGSEET

SEQ ID NO: 9 - DNA, 393 bp, Artificial
  Analog #10A
aacgatcgccacatgattagaatgcgtgagcttatagatgtggttgatcagctgaaaaattatgtgaatgagttggtccctgaatttctgccagctccagaagatatcgagacaaactgtgagtggtcagcttttacctgttttcagaaggcccaactacgctcagcaaatacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaattcctagaaagatacaaatcacttctccaaaagatggtgcatcagcatctgtcctccagaacacacggaagtgaagagtcc

SEQ ID NO: 10 - protein, 131 aa, Artificial
  Analog #10A
NDRHMIRMRELIDVVDQLKNYVNELVPEFLPAPEDIETNCEWSAFTCFQKAQLRSANTGNNERIIQVSIKKLKRKPPTTNAGRRNKHRLTCPSCDTYEKKPPKEFLERYKSLLQKMVHQHLSSRTHGSEES

SEQ ID NO: 11 - DNA, 399 bp, Artificial
  Analog #10B
caaggtaacgatcgccacatgattagaatgcgtgagcttatagatgtggttgatcagctgaaaaattatgtgaatgagttggtccctgaatttctgccagctccagaagatatcgagacaaactgtgagtggtcagcttttacctgttttcagaaggcccaactacgctcagcaaatacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaattcctagaaagatacaaatcacttctccaaaagatggtgcatcagcatctgtcctccagaacacacggaagtgaagagtcc

SEQ ID NO: 12 - protein, 133 aa, Artificial
  Analog #10B
QGNDRHMIRMRELIDVVDQLKNYVNELVPEFLPAPEDIETNCEWSAFTCFQKAQLRSANTGNNERIIQVSIKKLKRKPPTTNAGRRNKHRLTCPSCDTYEKKPPKEFLERYKSLLQKMVHQHLSSRTHGSEES

SEQ ID NO: 13 - DNA, 399 bp, Artificial
  Analog #4A
caaggtaacgatcgccacatgattagaatgcgtgagcttatagatgtggttgatcagctgaaaaattatgtgaatgacttggtccctcagtttctgccagctccagaagatatcgagacaaactgtgagtggtcagcttttacctgttttcagaaggcccaactaaagtcagcacagacaggaaacaatgaaaggataatccaggtatcaattaaaaagctgaagaggaaaccacctaccacaaatgcagggagaagaaacaaacacagactaacatgcccttcatgtgatacctatgagaaaaaaccacccaaagaattcctagaaagatacaaatcacttctccaaaagatggtgcatcagcatctgtcctccagaacacacggaagtgaagagacc

SEQ ID NO: 14 - protein, 133 aa, Artificial
  Analog #4A
QGNDRHMIRMRELIDVVDQLKNYVNDLVPQFLPAPEDIETNCEWSAFTCFQKAQLKSAQTGNNERIIQVSIKKLKRKPPTTNAGRRNKHRLTCPSCDTYEKKPPKEFLERYKSLLQKMVHQHLSSRTHGSEET

SEQ ID NO: 15 - protein, 31 aa, Homo sapiens
MASSPGNMERIVICLMVIFLGTLVHKSSSQG

SEQ ID NO: 16 - protein, 131 aa, Homo sapiens
QDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 17 - protein, 29 aa, Homo sapiens
MASSPGNMERIVICLMVIFLGTLVHKSSS

SEQ ID NO: 18 - DNA, 399 bp, Homo sapiens
caaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 19 - protein, 133 aa, Homo sapiens [sequence omitted]

(19) United States
(12) Patent Application Publication
(10) Pub. No.: US 2005/0124044 A1
(43) Pub. Date: Jun. 9, 2005

(54) Interleukin-21 analogs

(76) Inventors: Mark R. Cunningham, Kennett Square, PA (US); George A. Heavner, Malvern, PA (US); Jinquan Luo, Malvern, PA (US); Xiao-yu R. Song, West Chester, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/867,992

(22) Filed: Jun. 15, 2004

Related U.S. Application Data
(60) Provisional application No. 60/479,772, filed on Jun. 19, 2003.

(51) Int. Cl. C12P 21/02; C07H 21/04; C07K 14/54

(52) U.S. Cl. 435/69.5; 435/320.1; 435/325; 530/351; 536/23.5

(57) ABSTRACT

Isolated polynucleotides encoding mature interleukin-21, interleukin-21 analogs, polypeptides obtainable from the polynucleotides and uses are disclosed.

### FIELD OF THE INVENTION

The present invention relates to mature interleukin-21, interleukin-21 analogs and the polynucleotides encoding them.

### BACKGROUND OF THE INVENTION

Cytokines, the family of bioactive proteins and polypeptides synthesized by white blood cells and virtually all other nucleated cells, are secreted in response to microbes and other antigens, as well as environmental stimuli. They mediate diverse biological processes that are required for the maintenance of homeostasis and host defense. These processes include immune responses, inflammation, cell growth, tissue repair, fibrosis and angiogenesis. Cytokines play critical roles in host defense against pathogens and provide links between innate and adaptive immunity. They also regulate the magnitude and the nature of immune responses by influencing the growth and differentiation of immune cells.

The cytokine interleukin 21 (IL-21) has been identified by Parrish-Novak et al. in Nature 408, 57-63 (2000). Expression of human IL21 (huIL-21) in normal tissues was not detectable by Northern analysis. However, upon quantitative RT-PCR analysis, an increase in IL-21 mRNA level was detected in phorbol-12-myristate-13-acetate/ionomycin-activated or anti-CD3-treated human peripheral T cells suggesting that IL-21 may be used by T cells to stimulate effector cell function (Parrish-Novak et al., supra). In contrast, no IL-21 expression was detected on CD19⁺ B cells and CD 14⁺ monocytes (Parrish-Novak et al., supra).

The sequence of hIL-21 cDNA contains an open reading frame that encodes a polypeptide precursor of 162 amino acids. The signal peptidase cleavage rules predict a cleavage site after Gly31. The mature polypeptide is a soluble monomeric non-glycosylated protein with a predicted relative molecular mass of 15 kDa and consists of a 131-residue four-helix-bundle cytokine domain with significant homology to IL-2, IL-4, and IL-15, which also share a common C subunit receptor (Asao et al., J. immunol. 167, 1-5 (2001); Sugamura and Asao, Adv. Immunol. 59, 225 (1995); and Grabstein et al., Science 264, 965-968 (1994)). huIL-21 and huIL-15 share two pairs of cysteine residues in identical positions, one pair that is conserved in IL-2, IL-4, and granulocyte-macrophage colony-stimulating factor (GM-CSF) and one that is unique to IL-21 and IL-15. This structure consistency, together with the relatively high degree of amino-acid homology, indicates that IL-15 is the closest structural relative of IL-21. In general, cytokines are predicted to have a four-alpha helix structure, with helices A, C and D being most important in ligand-receptor interactions and more highly conserved among members of this family.

In vitro studies showed that IL-21 induces CD34⁺ bone marrow progenitor cell proliferation in combination with IL-15 (Parrish-Novak et al., supra). In addition, IL-21 also enhances effector function of the CD56⁺CD16^{bright} cells in the presence of Flt3L and IL-15. Moreover, NK cells cultured with IL-21 exhibit enhanced lytic activity on K562 target cells, although the effect of IL-21 is not quite as pronounced as that of IL-2 or IL-15. IL-21 also stimulates the proliferation of mature B cells when co-stimulated with anti-CD40 and T cells when co-stimulated with anti-CD3. In addition, IL-21 acts in concert with IL-2, IL-15, and IL-7 to enhance T cell proliferation, either with or without anti-CD3 stimulation (Parrish-Novak et al., supra). Recent studies showed that IL-21 enhances cytotoxic activity and IFNγ production by activated murine NK cells but does not sustain their viability. Moreover, IL-21 blocks IL-15-induced expansion of resting NK cells. In contrast, IL-21 enhances the proliferation, IFNγ production and cytotoxic function of antigen specific CD8⁺ effector T cells suggesting that IL-21 promotes the transition between innate and adaptive immunity (Kasaian et al., Immunity 16, 559-569 (2002)). In addition, 11-21 can inhibit antigen-specific and IL-4-induced IgE production and IL-21R deficient mice exhibited higher levels of IgE after immunization as compared with wild type counterparts (Suto et al., Blood 100, 4565-4573 (2002); Ozaki et al. Science 298, 1630-1634 (2002)). Finally, IL-21 is also a growth and survival factor for human myeloma cells (Brenne et al., Blood 99, 3756-3762 (2002)). Therefore, IL-21 seems to play an important role in regulating the immune system and may become a therapeutic target for various immune-mediated inflammatory disorders, allergic disorders, as well as cancers and infectious diseases. Thus, there is a need to generate a panel of monoclonal antibodies that will recognize different epitopes on IL-21 and could subsequently become potential therapeutics or diagnostics for a variety of diseases or research reagents for discovering new therapies. Novel analogs of huIL-21 can be used for the generation of antibodies that recognize IL-21. The use of these analogs to generate antibodies by immunization, phage panning or other techniques will result in antibodies that recognize specific regions of huIL-21. Analog IL-21 proteins can potentially provide enhanced properties, such as increased or modified biological half lives, modified biological activities, enhanced immunogenicity for generating antibodies, increased stability or expression, and the like.

### BRIEF DESCRIPTION OF THE DRAWINGS

FIG. 1 shows stimulation of human NK-92 cells by a huIL-21 analog and wild type huIL-21.

FIG. 2 shows an amino acid sequence alignment of huIL-21 analogs to the predicted mature form native sequence of huIL-21.

### SUMMARY OF THE INVENTION

One aspect of the invention is an isolated polynucleotide comprising a polynucleotide having the sequence shown in SEQ ID NO: 1, 3, 5, 7, 9, 11 or 13 or a complementary sequence.

Another aspect of the invention is an isolated polynucleotide comprising a polynucleotide encoding the amino acid sequence shown in SEQ ID NO: 2, 4, 6, 8, 10, 12 or 14 or a complementary sequence.

Another aspect of the invention is an isolated polypeptide comprising a polypeptide having the sequence shown in SEQ ID NO: 2, 4, 6, 8, 10, 12 or 14.

Another aspect of the invention is an isolated mature huIL-21 having the amino acid sequence shown in SEQ ID NO: 19.

Yet another aspect of the invention is an isolated polynucleotide encoding mature huIL-21 and having a sequence shown in SEQ ID NO: 18.

### DETAILED DESCRIPTION OF THE INVENTION

All publications, including but not limited to patents and patent applications, cited in this specification are herein incorporated by reference as though fully set forth. The present invention provides isolated analogs of IL-21. In particular, the invention provides huIL-21 analog polypeptides and polynucleotides. The polypeptides of the invention are related by amino acid sequence homology to the polypeptides having the sequences set forth in SEQ ID NO: 2, 4, 6, 8, 10, 12 or 14. The invention especially provides huIL-21 analogs having the amino acid sequences set forth in SEQ ID NOs: 2, 4, 6, 8, 10, 12 or 14 and polynucleotides encoding them including, but not limited to, polynucleotides having the sequences set forth in SEQ ID NOs: 1, 3, 5, 7, 9, 11 or 13 or their complementary sequences. The invention further provides for equivalent fragments and variants of huIL-21 analogs, as well as encoding or complementary nucleic acids, vectors comprising a huIL-21 analog, host cells containing such vectors and methods of making and methods of use of such analogs, vectors or host cells.

One aspect of the invention provides biologically active variants of huIL-21 useful for generation and screening of antibodies against IL-21. Anti-IL-21 antibodies are useful as therapeutic agents, diagnostic agents or research reagents. The present invention further provides for equivalent isolated polypeptides that: (a) comprise or consist of an amino acid sequence which has at least 95% identity, most preferably 97-99% or exact identity, to the entire amino acid sequence shown in SEQ ID NO: 2, 4, 6, 8, 10, 12 or 14; (b) is encoded by an isolated polynucleotide comprising or consisting of a polynucleotide sequence that has at least 95% identity, most preferably 97-99% or exact identity, to the entire nucleotide sequence shown in SEQ ID NO: 1, 3, 5, 7, 9, 11 or 13; or (c) is encoded by an isolated polynucleotide comprising or consisting of a polynucleotide sequence encoding a polypeptide which has at least 95% identity, most preferably 97-99% or exact identity, to the entire amino acid sequence shown in SEQ ID NO: 2, 4, 6, 8, 10, 12 or 14.

Values for % identity can be obtained from amino acid and nucleotide sequence alignments generated using the default settings for the AlignX component of Vector NTI Suite 8.0 (Informax, Frederick, Md.).

The polypeptides of the invention include a polypeptide having an amino acid sequence as set forth in SEQ ID NO: 2, 4, 6, 8, 10, 12 or 14 as well as equivalent polypeptides and fragments that have the biological activity of huIL-21 and have at least 95% identity to a polypeptide having the amino acid sequence shown in SEQ ID NO: 2, 4, 6, 8, 10, 12 or 14 and include portions of the polypeptide generally containing at least 15-50 amino acids. Exemplary embodiments of the invention are the polypeptides having the amino acid sequences set forth in SEQ ID NO: 8, 10, 12 or 14.

In the invention, the polypeptides having the amino acid sequences set forth in SEQ ID NO: 8 or 10 have two additional N-terminal amino acids as set forth in SEQ ID NOs: 12 and 14, respectively. Exemplary nucleotide sequences encoding these polypeptides are set forth in SEQ ID NOs: 11 and 13, respectively. The present inventors have discovered that mature IL-21 does not have a mass corresponding to an N-terminal residue of Gln-32 (as predicted by Novak et al. in U.S. Pat. No. 6,307,024) but instead has a mass of 16,815.8 Da corresponding to Gln-30 as the actual N-terminal residue. See Example 2 below. Thus, the actual secretory signal sequence includes amino acid residues 1 (Met) to 29 (Ser) (SEQ ID NO: 17) and the actual mature polypeptide includes amino acid residues 30 (Gln) to 162 (Ser) (residues 1 to 133 in SEQ ID NO: 19). Thus, another aspect of the invention is an isolated mature human interleukin-21 having the sequence shown in SEQ ID NO: 19 and polynucleotides encoding it such as that having the sequence shown in SEQ ID NO: 18.

A “fragment” is a variant polypeptide having an amino acid sequence that is entirely the same as part but not all of any amino acid sequence of any polypeptide of the invention. Fragments can include, e.g., truncation polypeptides having a portion of an amino acid sequence as shown in amino acid sequence shown in SEQ ID NO: 2, 4, 6, 8, 10 or 12, or of variants thereof, such as a continuous series of residues that includes a heterologous amino- and/or carboxy-terminal amino acid sequence. Degradation forms of the polypeptides of the invention produced by or in a host cell are also included. Other exemplary fragments are characterized by structural or functional attributes such as fragments that comprise alpha-helix or alpha-helix forming regions, beta-sheet or beta-sheet forming regions, turn or turn-forming regions, coil or coil-forming regions, hydrophilic regions, hydrophobic regions, alpha-amphipathic regions, beta-amphipathic regions, flexible regions, surface-forming regions, substrate binding regions, extracellular regions and high antigenic index regions.

Further exemplary fragments include an isolated polypeptide comprising an amino acid sequence having at least 15, 20, 30, 40, 50 or 100 contiguous amino acids from the amino acid sequence set forth in SEQ ID NO: 2, 4, 6, 8, 10, 12 or 14, or an isolated polypeptide comprising an amino acid sequence having at least 15, 20, 30, 40, 50 or 100 contiguous amino acids truncated or deleted from the amino acid sequence set forth in amino acid sequence shown in SEQ ID NO: 2, 4, 6, 8, 10, 12 or 14.

The present invention further provides for equivalent isolated polynucleotides that comprise or consist of (a) a polynucleotide sequence which has at least 95% identity, most preferably 97-99% or exact identity, to the entire nucleotide sequence shown in SEQ ID NO: 1, 3, 5, 7, 9, 11 or 13; or (b) a polynucleotide encoding a polypeptide sequence that has at least 95% identity, most preferably 97-99% or exact identity, to the entire polypeptide sequence shown in SEQ ID NO: 2, 4, 6, 8, 10, 12 or 14.

The polynucleotides of the invention include a mature polypeptide coding sequence having a nucleotide sequence as set forth in SEQ ID NO: 1, 3, 5, 7, 9, 11 or 13.

Exemplary embodiments of the invention are the polynucleotides having the nucleotide sequences set forth in SEQ ID NO: 7, 9, 11 or 13.

The invention also provides a mature polypeptide coding sequence or a fragment thereof in reading frame with another coding sequence, such as a sequence encoding a leader or secretory sequence, a pre- or pro- or prepro-protein sequence. The polynucleotides of the invention may also contain at least one non-coding sequence, such as transcribed but not translated sequences, termination signals, ribosome binding sites, Kozak sequences, mRNA stabilizing sequences, introns and polyadenylation signals. The polynucleotide sequences may also contain additional sequences encoding additional amino acids. These additional polynucleotide sequences may, for example, encode a marker sequence such as a hexa-histidine peptide, as described in Gentz et al., Proc. Natl. Acad. Sci. (USA) 86, 821-824 (1989) or the HA peptide tag as described in Wilson et al., Cell 37, 767 (1984) which facilitate the purification of fused polypeptides. Polynucleotides of the invention can also include structural gene polynucleotides and associated gene expression control sequences.

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

For recombinant production of the polypeptides of the invention, host cells can be genetically engineered to incorporate expression systems or portions thereof and polynucleotides of the invention. Introduction of a polynucleotide into a host cell can be effected by methods well known to those skilled in the art from laboratory manuals such as Davis et al., Basic Methods in Molecular Biology, 2^{nd} ed., Appleton & Lange, Norwalk, Conn. (1994) and Sambrook et al., Molecular Cloning: A Laboratory Manual, 3^{rd} ed., Cold Spring Harbor Laboratory Press, Cold Spring Harbor, N.Y. (2001). These methods include calcium phosphate transfection, DEAE-Dextran mediated transfection, transvection, microinjection, cationic lipid-mediated transfection, electroporation, transduction, scrape loading, ballistic introduction and infection.

Representative examples of hosts include Archaea cells; bacterial cells such as streptococci, staphylococci, enterococci, E. coli, streptomyces, cyanobacteria, B. subtilis and S. aureus; fungal cells such as Kluveromyces, Saccharomyces, Basidomycete, Canidida albicans or Aspergillus; insect cells such as Drosophila S2 and Spodoptera Sf9; animal cells such as CHO, COS, HeLa, C127, 3T3, BHK, 293, CV-1, Bowes melanoma and myeloma; and plant cells, such as gymnosperm or angiosperm cells.

A great variety of expression systems can be used to produce the polypeptides of the invention. Such systems include chromosomal-, episomal- and virus-derived vectors such as vectors derived from bacterial plasmids, bacteriophage, transposons, yeast episomes, insertion elements, yeast chromosomal elements, baculoviruses, papova viruses such as SV40, vaccinia viruses, adenoviruses, fowl pox viruses, pseudorabies viruses, picronaviruses and retroviruses and vectors derived from combinations thereof, such as cosmids and phagemids. The expression system constructs may contain control regions that regulate or cause expression. Generally, any system or vector suitable to maintain or propagate polynucleotides and/or to express polypeptides in a host may be used for expression. An appropriate DNA sequence may be inserted into the expression system by any of a variety of techniques well known to those skilled in the art, such as, e.g., those set forth in Sambrook et al., supra.

In eukaryotic expression systems, polypeptides of the invention can be secreted into the lumen of the endoplasmic reticulum or extracellular environment by inclusion of appropriate secretion signals such as a signal peptide or leader sequence. These signals may be heterologous or endogenous to huIL-21 such as those listed in SEQ ID NO: 15 (predicted) or SEQ ID NO: 17 (actual).

The polypeptides of the present invention may also be produced by chemical synthesis such as solid phase peptide synthesis on an automated peptide synthesizer, using known amino acid sequences or amino acid sequences derived from the DNA sequence of the polynucleotides of the invention. Such techniques are well known to those skilled in the art.

Polypeptides of the invention can be recovered and purified from recombinant cell cultures by well-known methods including ammonium sulfate or ethanol precipitation, acid extraction, high-performance liquid chromatography, anion or cation exchange chromatography, phosphocellulose chromatography, hydrophobic interaction chromatography, affinity chromatography, hydroxyapatite chromatography and lectin chromatography. Well-known techniques for refolding protein may be employed to regenerate an active conformation when the protein is denatured during isolation and/or purification.

The polynucleotides and polypeptides of the invention comprising at least one epitope of IL-21 can be used to produce polyclonal or monoclonal antibodies. These analogs may exhibit increased binding efficiency to IL-21 receptor and/or be more immunogenic than wild type IL-21. Techniques for making murine, chimeric, humanized and fully human monoclonal antibodies using protein or nucleic acid immunization are known to those skilled in the art.

The polynucleotides and polypeptides of the invention are also useful for assaying a medium for the presence of a substance that modulates IL-21 protein function by affecting the binding of an IL-21 analog protein to cellular binding partners such as the IL-21 receptor. Examples of modulators include polypeptides or small organic molecules.

Mature IL-21 or its analogs can be used to modulate, i.e., increase or decrease, immune cell activity and/or number such as the activity and/or number of T cells (CD4⁺, CD8⁺ and mature T cells), NK cells (resting or mature), B cells, dendritic cells, macrophages, megakaryocytes or a population of immune cells. Moreover, mature IL-21 or its analogs can be used to dampen IgE production in allergic diseases as well as asthma and other parasitic diseases mediated by IgE. Since IL-21 can inhibit the production of IFNγ from developing Th1 cells (Wurster et al., J. Exp. Med 196, 969-977, (2002)), mature IL-21 or its analogs can also be used to treat various kinds of immune-mediated inflammatory diseases that are dependent on IFNγ such as multiple sclerosis. In addition, mature IL-21 or its analogs can be used to enhance immune responses to a cancer or infectious disease. Mature IL-21 or its analogs can be used alone or in combination with an antigen as an adjuvant to treat or prevent various cancers such as solid tumors, soft tissue tumors (such as lymphoma or leukemia) and metastatic lesions. Further, mature IL-21 or its analogs can be used to treat or prevent infectious disorders including bacterial, viral and parasitic disorders.

The mode of administration for therapeutic use of the polypeptides of the invention may be any suitable route which delivers the agent to the host. The polypeptides and their pharmaceutical compositions of these agents are particularly useful for parenteral administration, i.e., subcutaneously, intramuscularly, intradermally, intravenously or intranasally.

Polypeptides of the invention may be prepared as pharmaceutical compositions containing an effective amount of the binding agent as an active ingredient in a pharmaceutically acceptable carrier. An aqueous suspension or solution containing the binding agent, preferably buffered at physiological pH, in a form ready for injection is preferred. The compositions for parenteral administration will commonly comprise a solution of the binding agent of the invention or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the polypeptides of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of a polypeptide of the invention. Similarly, a pharmaceutical composition of the invention for intravenous infusion could be made up to contain about 250 ml of sterile Ringer's solution, and about 1 mg to about 30 mg and preferably 5 mg to about 25 mg of a polypeptide of the invention. Actual methods for preparing parenterally administrable compositions are well known or will be apparent to those skilled in the art and are described in more detail in, for example, Remington, the Science and Practice of Pharmacy, 19th ed., Mack Publishing Company, Easton, Pa. (1995).

The polypeptide of the invention, when in a pharmaceutical preparation, can be present in unit dose forms. The appropriate therapeutically effective dose can be determined readily by those of skill in the art. A determined dose may, if necessary, be repeated at appropriate time intervals selected as appropriate by a physician during the treatment period.

The polypeptides of the invention can be lyophilized for storage and reconstituted in a suitable carrier prior to use. This technique has been shown to be effective with conventional protein preparations and art-known lyophilization and reconstitution techniques can be employed.

The present invention will now be described with reference to the following specific, non-limiting examples.

### EXAMPLE 1

Human IL-21 Analog Design and Testing

A molecular model of huIL-21 was created with the InsightII software suite (Accelrys, San Diego, Calif.). Each amino acid in the model was examined visually and potential substitutions considered on the basis of retention of hydrophobic, steric and hydrogen-bonding characteristics of the parent amino acids. A molecular model of each proposed analog was made using the same conditions used to create the parent molecule model. These structures were evaluated and structures with any of the following properties were not considered further:
a structure of significantly higher energy as calculated by InsightII;
significant alterations in the backbone structure;
significant changes in the surface exposed residues;
significant changes of the hydrophobicity of the surface; or
alterations in hydrogen-bonding patterns.

Various combinations of acceptable amino acid substitutions were then modeled further and evaluated as previously described. Of the combinations considered, eight analogs were generated.

DNA encoding each of the eight analogs was obtained synthetically and cloned into a mammalian cell expression vector containing the CMV promoter and the bovine growth hormone polyA transcriptional control sequences and evaluated for their ability to generate a secreted protein. Polypeptides produced by the expression vector contained an N-terminal secretory signal sequence (SEQ ID NO: X) from wild type IL-21 and a C-terminal hexa-histidine tag. Briefly, HEK 293E cells were transfected with the expression vectors using a cationic lipid reagent to facilitate DNA uptake by the cells. After 24 hours, the cells were placed in a serum-free medium formulation and grown for 4 days. Following this period, the conditioned medium was recovered and subjected to immobilized metal affinity chromatography (IMAC) using TALON™ resin (CLONTECH Laboratories, Inc., Palo Alto, Calif.) to purify His-tagged proteins. Any proteins bound to the column were eluted using EDTA and subjected to Coomassie-stained SDS PAGE and anti-His Western blot. Analysis of visible bands on the SDS PAGE gels and anti-His Western blots indicated that human IL-21 analog #1, 2, 3, and 4 (SEQ ID NOs: 2, 4, 6 and 8, respectively) were secreted (data not shown). Biological activity of analog #4 (SEQ ID NOs: 7 and 8) was determined in an NK-92 cell proliferation assay. This analog was expressed at IL scale and purified using IMAC TALON™. Human NK-92 cells were cultured in 96-well flat bottom tissue culture plates (white plate and clear bottom, VWR, Bridgeport, N.J.). 100 μL of cells were plated per well at a density of 1×10⁵ cells per ml in MEM (Invitrogen, Carlsbad, Calif.) supplemented with 12.5% FBS (JRH Bioscience, Lenexa, Kans.), 12.5% horse serum, 0.2 mM inositol, 0.02 mM folic acid, 100 gM beta-mercaptoethanol (Sigma, St. Louis, Mo.), 10 ng/ml of hIL-2 and hIL-15 (PeproTech, Rocky Hill, N.J.). The cells were cultured in triplicates, washed twice with medium and assayed for proliferation in the presence of serially diluted purified IL-21, IMAC TALON™ purified IL-21 analog #4 or control protein (murine tissue factor variant) for 72 hrs at 37° C., 5% CO,. Per kit directions (Packard, Boston, Mass.), the cells were then lysed and ATP-lite substrate was added. Luminescence was measured by using a Topcount plate reader (Packard).

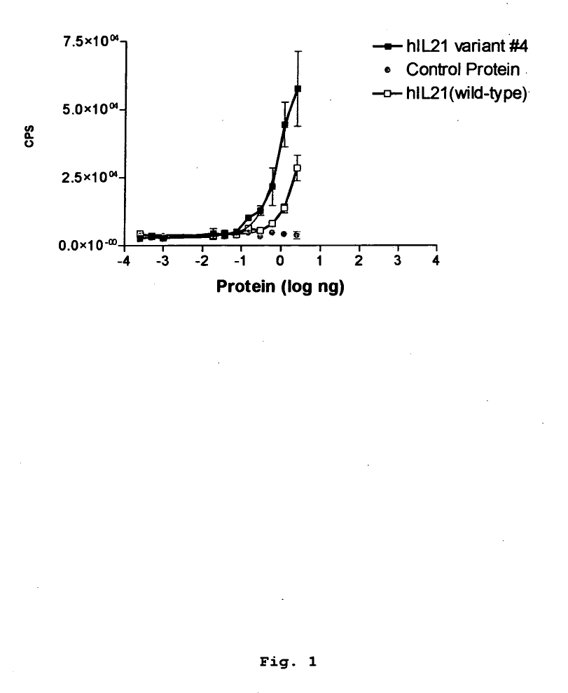

The results shown in FIG. 1 indicated that both huIL-21 variant #4 and wild-type protein promote the proliferation of NK-92 cells in a dose dependent fashion. The EC₅₀ for hIL-21 variant #4 is 1.014 ng/ml and that of wild-type hIL-21 protein ranges between 1 ng/ml and 4.6 ng/ml for different lots. Each data point represents the mean ± SD of triplicates.

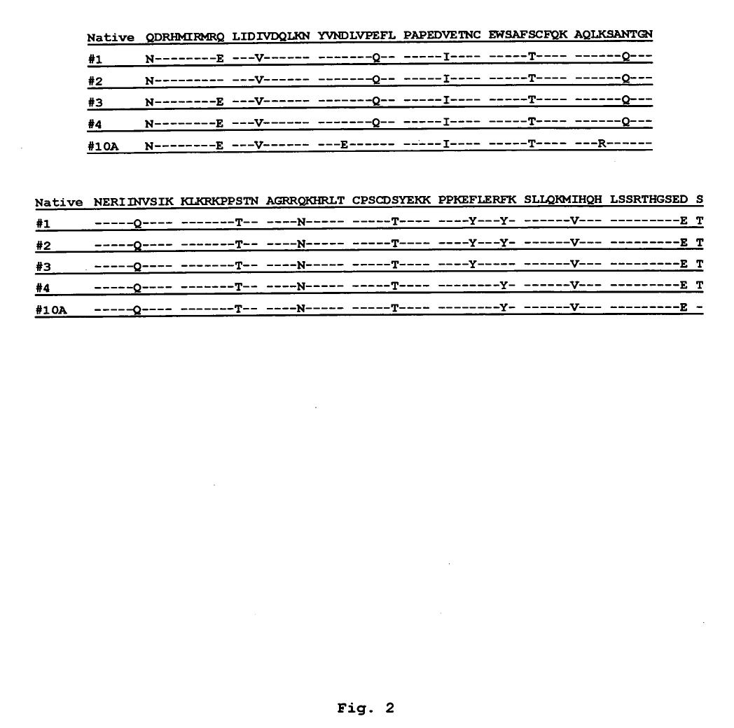

Analog #4 was used as a template for further rounds of variant construction. The original mutations were grouped into four families depending upon surface and internal positioning. For each family, two sets of mutations were made either to revert amino acids back to wild-type sequence or change amino acids to an alternative sequence. Nine variants were expressed and protein secretion characterized as described above. The results (not shown) indicated that huIL-21 analog 10A was expressed and secreted at slightly reduced levels compared to wild-type huIL-21. The amino acid sequence alignment of huIL-21 analog nos. 1, 2, 3, 4 and 10A (SEQ ID NOs: 2, 4, 6, 8 and 10, respectively) compared to the predicted mature form native sequence of huIL-21 are shown in FIG. 2.

### EXAMPLE 2

SELDI-TOF Mass Spectrometry of huIL-21

Purified recombinant wild-type human IL-21 protein was analyzed by Surface Enhanced Laser Desorption Ionization (SELDI) Time-of-Flight (TOF) mass spectrometry. Briefly, 3 μL of huIL-21 (≧0.1 mg/mL) was spotted on to a C18 hydrocarbon derivatized LDI-TOF solid sample support H4 chip (Ciphergen Biosystems, Inc., Fremont, Calif.) and allowed to dry at room temperature. The sample was then washed on the chip three times with 3 μL Milli-Q H₂O (Millipore Corporation, Billerica, Mass.) and 1 μL of saturated sinapinic acid (Sigma-Aldrich Co., St. Louis, Mo.) in 50% acetonitrile with 1% trifluoroacetic acid was applied to the IL-21 spot and allowed to dry at room temperature. The mass spectrum was acquired on a SELDI spectrometer (Ciphergen Biosystems, Inc.) to obtain the m/z values for the protein sample. The mass measured for recombinant IL-21 was 16,815.8 Da, suggesting a mature protein beginning at position 30 of the precursor sequence (Gln-Gly-Gln-Asp-Arg-His- vs Gln-Asp-Arg-His-).

The present invention now being fully described, it will be apparent to one of ordinary skill in the art that many changes and modifications can be made thereto without departing from the spirit or scope of the appended claims.

1. An isolated polynucleotide comprising a polynucleotide having the sequence shown in SEQ ID NO: 1, 3, 5, 7, 9, 11 or 13 or a complementary sequence.

2. An isolated polynucleotide comprising a polynucleotide encoding the amino acid sequence shown in SEQ ID NO: 2, 4, 6, 8, 10, 12 or 14 or a complementary sequence.

3. An isolated polypeptide comprising a polypeptide having the sequence shown in SEQ ID NO: 2, 4, 6, 8, 10, 12 or 14.

4. Isolated mature human interleukin-21 (huIL-21) having the amino acid sequence shown in SEQ ID NO: 19.

5. An isolated polynucleotide encoding the mature huIL-21 of claim 4.

6. The isolated polynucleotide of claim 5 having a sequence shown in SEQ ID NO: 18.

7. A vector comprising the isolated polynucleotide of claim 1, 2 or 5.

8. An isolated host cell comprising the vector of claim 7.

9. A process for producing a polypeptide comprising culturing the host cell of claim 8 under conditions sufficient for production of the polypeptide.
